(19) United States
(12) Patent Application Publication
(10) Pub. No.: US 2014/0085421 A1
(43) Pub. Date: Mar. 27, 2014

(54) ENDOSCOPE HAVING 3D FUNCTIONALITY

(71) Applicants: Rainer Kuth, Hochstadt (DE); Philip Mewes, Nurnberg (DE); Helmut Neumann, Erlangen (DE); Markus Neurath, Erlangen (DE); Anton Schick, Velden (DE)

(72) Inventors: Rainer Kuth, Hochstadt (DE); Philip Mewes, Nurnberg (DE); Helmut Neumann, Erlangen (DE); Markus Neurath, Erlangen (DE); Anton Schick, Velden (DE)

(21) Appl. No.: 13/883,319

(22) PCT Filed: Aug. 23, 2011
(86) PCT No.: PCT/EP2011/064450

§ 371 Date: Dec. 11, 2013

(30) Foreign Application Priority Data
Nov. 4, 2010 (DE) 102010050227.8

(51) Int. Cl. A61B 1/00

(52) U.S. Cl. CPC A61B 1/00009
USPC 348/45; 348/65

(57) ABSTRACT

An endoscope for quantitatively determining dimensions of an object in a cavity is provided. The endoscope has a projector connected to a light guide that can be introduced through the instrument channel. In the introduced position the projector projects a pattern at a defined illumination angle onto the object to be examined. The endoscope has a camera that captures the projected pattern at a fixed viewing angle in a distorted from corresponding to the object. The distorted form is used for quantitatively determining dimensions of the object by a triangulation method.

### CROSS REFERENCE TO RELATED APPLICATIONS

This application is the US National Stage of International Application No. PCT/EP2011/064450 filed Aug. 23, 2011 and claims benefit thereof, the entire content of which is hereby incorporated herein by reference. The International Application claims priority to the German application No. 10 2010 050 227.8 DE filed Nov. 4, 2010, the entire contents of which is hereby incorporated herein by reference.

### FIELD OF INVENTION

The present invention relates to an endoscope having 3D functionality.

### BACKGROUND OF INVENTION

The extent of diseased structures is currently assessed in endoscopy on the basis of estimations. A precise dimension of diseased structures is not possible with currently available methods. On account of the two-dimensional representation of the endoscopic image, only rough estimations relating to surface details were previously possible. A volume measurement is not possible. With many gastrointestinal diseases, these parameters nevertheless play an important role for further diagnostics and therapy. Examples are herewith the so-called Barrett's esophagus, colorectal polyps or chronic inflammatory bowel disease.

Barrett's esophagus refers to precancerous cells at the transition from the esophagus to the stomach, which relates to approximately 10% of patients with so-called reflux disease (main symptom: heartburn). Over the last 25 years there has been a drastic increase in reflux diseases and consecutively also esophageal cancer. Contrary to advanced findings, which are characterized by an extremely poor prognosis (frequently only a few months), the early forms of Barrett and esophageal cancer can be relatively effectively treated using minimally invasive means (without an operation). Their respective extent plays an important role in the early diagnosis of these changes. The possibility of measuring a volume would open up new perspectives in terms of early detection and prevention of this disease.

The association of colorectal polyps with bowel cancer has long been known. National and international guidelines recommend the earliest possible removal of these polyps in order to prevent the development of bowel cancer. Nevertheless not all polyps lead to bowel cancer. The so-called risk polyps (polyps with a high risk of developing bowel cancer) are in this way characterized on the basis of their size and their surface structure (the so-called pit pattern). On grounds of cost, attempts are made to detect and remove only these polyps as far as possible. With currently available methods, a precise characterization of the polyps is however not possible so that all detected polyps must generally also be removed. The resulting high costs of removal, the often resulting hospitalization of the patient and the subsequent histopathological assessment may therefore be spared in the future thanks to a more accurate 3-dimensional characterization of the polyps and the polyp surface.

Chronic inflammatory bowel diseases represent a further important indication in respect of the endoscopic examination of the gastrointestinal tract. Affected patients suffer from a significant reduction in quality of life and are often subjected to medical treatment with numerous side affects. Furthermore, the patients in some cases demonstrate a significantly increased risk of developing cancer compared with the normal population. For this reason, the most precise description of the affected mucous membrane as possible is decisive for the endoscopic diagnostics of affected patients, which, on account of the already cited limitations of current endoscopic methods, is in some cases possible only to a very limited extent. A volume measurement of affected mucous membrane sections would allow for more precise diagnostics in patients and thus a more targeted therapy, control and provision.

Penetration of medical endoscopy by technical innovations from the field of 3D measuring technology, previously relatively minimal, allows the opportunity of realizing new and improved diagnosis options. A high acceptance is achieved by a simple but efficient expansion of existing endoscope technology.

Current approaches according to the prior art are based on the stereoscopic or photogrammetric approach, which provides for a high degree of effort on account of two parallel viewing optics, and presupposes clearly recognizable structures (natural features) of the object and presupposes an extremely high computing capacity in order to be able to operate as it were in real-time with a high image rate. If a surface has no identifiable structures, no 3D recordings can be made in these regions. The 3D acquisition is only partially possible. The principle of the afore-cited two parallel viewing optics can be inferred from FIG. 1. A further such approach can be inferred from the publication DE 44 24 114 C1 [1].

A further approach was pursued within the faculty of pattern recognition at the University of Erlangen-Nuremberg. This concerns a “3D hybrid system”, which records depth data by measuring the time the light takes to travel. The achievable resolution capacity is herewith nevertheless very restricted in all three spatial dimensions, since the precise measurement of the time the light takes to travel is required for each image pixel (camera pixel) and therefore presupposes a very specialized camera development. A resolution in the centimeter range can herewith be achieved under optimum preconditions.

Approaches involving resolving the challenges of 3D endoscopy by means of phase grid projection actually already existed in the 90's. This nevertheless presupposes the recording of a plurality of (at least 6) projected phase layers and thus a very complex projector, which has the possibility of changing the image. Furthermore, this solution must be realized within the specifications by the endoscopic boundary conditions, such as small dimensions and low weight. Furthermore, the frame rate must be very high to ensure that camera shake blurring does not result in a lack of measuring clarity.

Different technologies were put to the test in order to measure the third dimension. This also relates in particular to the afore-described method as claimed in the prior art such as
phase-encoded active triangulation
method for measuring the time the light takes to travel
confocal 3D method
laser scanning

Special attention was given to the method which stands out in terms of its many properties, the so-called “Color Coded Triangulation CCT”. The CCT was originally developed for the three-dimensional measurement of the human face for biometric use and was also used here in applications within the cosmetics industry.

The color-coded line pattern projected onto a face is clearly identified in FIG. 2. Since the projection direction and the viewing direction are different, the originally straight lines are deformed by the three-dimensional shape of the face. The third dimension is in turn calculated from this deformation. Further fields of application were concluded in the automobile industry in order to measure the wheel base during vehicle assembly and within the field of hearing device industry.

The publication DE 197 42 264 A1 [2] discloses an endoscope for the optical three-dimensional detection of objects with a recording facility for optically detecting the object, and an illumination facility for illuminating the object. The apparatus includes a pattern generation facility for generating an optical pattern, a deflecting facility with a first deflecting surface assigned to the recording facility and a second deflecting surface assigned to the pattern generation facility, as well as an elongated housing with an optical window, in which region the deflection facility is provided, wherein the recording facility, deflecting facility and pattern generation facility are arranged along the longitudinal axis of the housing.

### SUMMARY OF INVENTION

The object therefore underlying the present invention is to specify an endoscope having 3D functionality, which retains the familiar device surroundings during gastroscopy or colonoscopy and is embodied to be structurally compact, nevertheless enables a precise calculation of the 3-dimensional surface representation, a volume measurement and a determination of the exact extent of the viewed object.

This object is achieved by the features specified in the independent claim. Advantageous embodiments of the invention are specified in the further claims.

As a result
a projection facility can be introduced through the instrument channel, with which, in the introduced position, a pattern is projected onto the object to be examined at a defined illumination angle;
the camera captures this projected pattern at a fixed viewing angle in a distorted form corresponding to the object and
the distorted form is used by means of a triangulation method for quantitatively determining dimensions of the object
an endoscope is created, which retains the device surroundings during gastroscopy or colosocopy, can be easily handled and uses existing components such as camera or image guide for a further recording method.

The following advantages may additionally result:
) simple applicability with high effectiveness by multiple use of classic endoscope equipment;
ii) high acceptance of a new product, since familiar functionalities are retained;
iii) additional costs are relatively low.

The following advantages may result for specific embodiments of the invention if the light source is arranged on the side facing away from the examination side.
iv) there is no emission of heat in the viewing room.
v) the projection lens connected to a lightwave conductor does not require any additional electrical cabling in the instrument channel since the light source is at a distance.

This triangulation method realized according to the invention in an endoscope not only means that the topology of the 3D surface of the object and/or organ is known, but also in particular that this topology can be quantitatively determined in contrast to 2D endoscopy, in which distances between two objects which appear in the image are not known precisely.

Advantageously, anatomical or pathological landmarks are identified by means of image processing and accurate distances in the image are then shown. Optionally, the gastroenterologist can set individual markers in the respectively indicated image and then display the respective distances. Furthermore, surfaces can be quantified, e.g. complete surfaces of the stomach or volume of the stomach. Average depths of the gaps can also be evaluated and displayed.

It is also possible to measure dynamic 3D parameters, therefore the volume of the stomach can be measured as a function of time for instance, thereby giving importance to the diagnosis of gastric voiding disorders.

### BRIEF DESCRIPTION OF DRAWINGS

The invention is explained in more detail below with the aid of the drawing for instance, in which:

FIG. 1 shows a representation of a stereoscopic or photogrammetric approach with two parallel viewing optics according to the prior art;

FIG. 2 shows a color-coded line pattern on a face;

FIG. 3 shows a basic representation of an embodiment of the invention of an endoscope with CCT receiver.

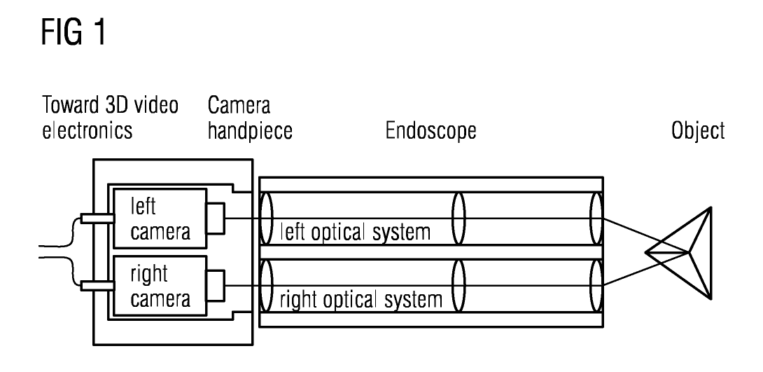

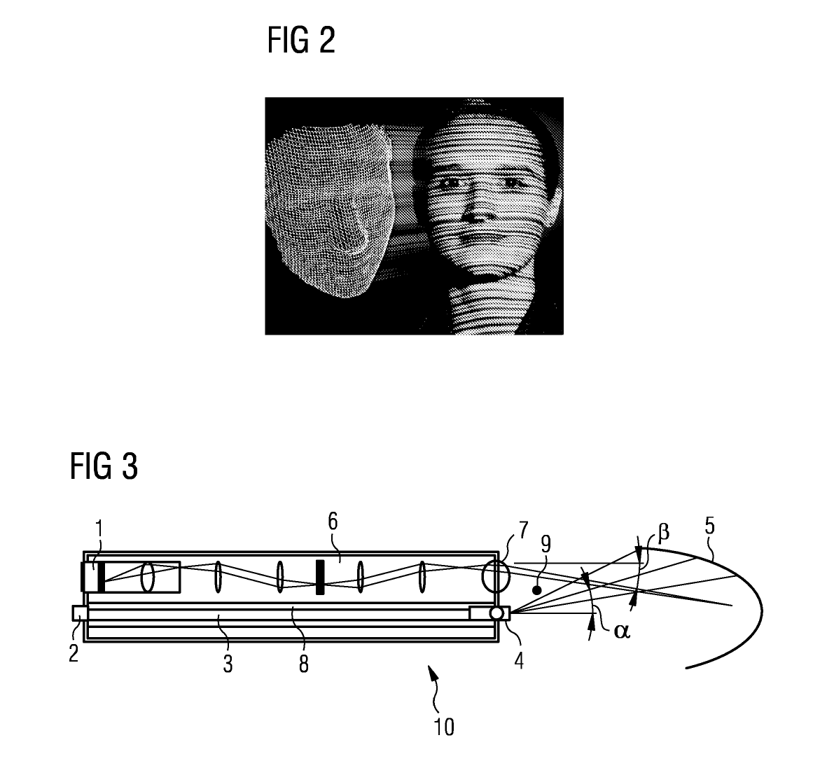

### DETAILED DESCRIPTION OF INVENTION

FIG. 3 shows a basic representation of an embodiment of the invention: a light source 2 arranged outside of or precisely on the endoscope, which is connected to a projection facility by way of a light guide 3, recording lens 7, wherein the received light beams are routed to a camera 1 via a lens system 6.

This medical-technical recording method which is realized in the endoscope 10 is based on classic endoscopy in conjunction with a triangulation method such as preferably the CCT (Color Coded Triangulation). A (colored) pattern is in the process projected at an illumination angle a onto the object 5 to be examined or measured and the (colored) pattern visible on the measuring object 5 is recorded at a viewing angle 0 by means of the camera 1. The illumination angle and viewing angle are determined in this way by the extent of the pattern in the projection space and/or in the viewing space. The projected pattern appears distorted on account of the form of the object 5 and the 3D form of the object is in turn reconstructed from this distortion by means of special algorithms. Since only one pattern is projected, the technical effort in the “region of the projector” is relatively low. This recording method can therefore also be realized in existing endoscopes 10, it is shake-proof and can be operated in real-time (30 Hz). Instead of a colored pattern for CCT, a monochrome pattern or a sequence of patterns can also be projected and classical triangulation methods such as the phase-encoded active triangulation are used for the evaluation.

With “large” objects, which do not fit into the field of view of the camera 1 at the same time, a subarea of the 3D space is captured with each camera recording. By a number of recordings being made, which overlap in the subareas, large objects 5 can also be reconstructed in terms of their three-dimensional form by means of “data stitching”. Individual three-dimensional parts of a surface are combined in a 3D puzzle by means of the corresponding mathematical operations, until the overlapping is as optimal as possible.

Retaining the familiar device surroundings during gastroscopy or colonoscopy allows such an endoscope 10 to be retained in respect of all its relevant functions. The 3D functionality can be realized by the use of the instrument channel 8. This instrument channel 8 typically has a diameter of 2.5 mm to 4.5 mm. With diameters of this order of magnitude, an optical projection facility 4 can be takes as far as the tips of the endoscope 10. This projection facility 4 contains a slide, which has the projection structure for the pattern to be projected, an optic for the targeted supply of the white projection light from the light source 2 to the slide and a projection optic 4 which projects the pattern onto the object 5. The supply of light takes place by means of a flexible light conductor 3, which adjusts to the curvature of the endoscope. In order to realize the triangulation, use is additionally made of the already existing imaging optics 7 of the endoscope 10. All functions of the endoscope 10 are retained except for the availability of the channel during the 3D measurement (cleaning function, air supply, Bowden cable for movement of the endoscope tip etc.).

The projection facility 4 which can be introduced through the instrument channel can be embodied on the viewing side 9, as a function of the field of use of the endoscope 10, as follows:
a) a projection lens 4 is connected via a light guide 3 to a light source 2, which is arranged on the side of the endoscope 10 facing away from the viewing side.
b) the projection facility 4 includes light source 2 and projection lens 4 on the viewing side 9. The power supply can either be provided by means of a battery disposed in the projection facility or by an electrical line, which is found in the instrument channel 8. This line is introduced into the instrument channel 8 together with the projection facility 4.

Irrespective of the afore-cited embodiment of the projection facility 4, provision can be made on the observation side 9 for a mechanical apparatus, such that this engages in a defined manner on the observation side 9 upon introduction of the projection facility 4. The illumination angle a is as a result defined for the projection.

The camera 1 can be arranged, as shown in FIG. 3, similarly on the side facing away from the viewing side 9, wherein the pattern captured by the recording lens 7 is fed to the camera 1 by way of a lens system 6 or an image guide. An embodiment is also possible, where the camera 1 is arranged as a whole on the viewing side 9, but is not shown in the Figures.

### LIST OF REFERENCE CHARACTERS, GLOSSARY

1 camera
2 light source
3 light guide in the instrument channel
4 color pattern projector, projection lens; projection facility
5 object, object to be examined
6 lens system
7 recording lens, front lens
8 instrument channel
9 viewing side
10 endoscope; endoscope for gastroscopy, colonoscopy
α illumination angle
β viewing angle
CCT Color Coded Triangulation

### List of Cited Documents

[1] DE 44 24 114 C+
   <3D Video Endoscope>
Nuclear Research Center Karlsruhe GmbH
DE-76133 Karlsruhe
[2] DE 107 42 264 A1
   <Endoscope>
   Vosseler Erste Patentverwertungsgesellschaft mbH
DE-74613 Öhringen

1.-12. (canceled)

13. An endoscope for quantitatively determining variables of an object in a cavity, comprising:
an instrument channel;
a camera; and
a projector that can be introduced through the instrument channel for projecting a pattern onto the object to be examined in an introduced position,
wherein the camera is configured to capture the projected pattern in a distorted form corresponding to the object, and
wherein the variables of the object is quantitatively determined based on the distorted form by a triangulation method.

14. The endoscope as claimed in claim 13, wherein the projector is connected to a light source by a light guide and the pattern to be projected is formed by a slide disposed in the projector.

15. The endoscope as claimed in claim 14, wherein the light source is arranged at an end of the endoscope facing away from a viewing side.

16. The endoscope as claimed in claim 13, wherein the projector comprises a light source and the pattern to be projected is formed by a slide disposed in the projector.

17. The endoscope as claimed in claim 16, wherein the projector comprises a battery for supplying power to the light source.

18. The endoscope as claimed in claim 16, wherein the projector comprises an electrical line disposed in the instrument channel for supplying power to the light source.

19. The endoscope as claimed in claim 13, wherein the variables of the object is quantitatively determined by the triangulation method comprising:
calculating a 3-dimensional surface shape of the object,
calculating a surface content of a surface of the object,
measuring a volume of the object, or
determining an extent of the object in different directions.

20. The endoscope as claimed in claim 13, wherein the projected pattern is an encoded colored pattern.

21. The endoscope as claimed in claim 13, wherein the projected pattern is a monochrome pattern.

22. The endoscope as claimed in claim 13, wherein the projector projects a sequence of patterns.

23. The endoscope as claimed in claim 13, wherein the triangulation method is a Color Coded Triangulation method.

24. The endoscope as claimed in claim 13, further comprising a mechanical apparatus arranged at an end on a viewing side so that the projector engages into a predetermined position upon introduction.
